(19) United States
(12) Patent Application Publication
(10) Pub. No.: US 2018/0201662 A1
(43) Pub. Date: Jul. 19, 2018

(54) PEPTIDE FOR PROMOTING CELL MIGRATION AND/OR SKIN WOUND HEALING, PHARMACEUTICAL COMPOSITION CONTAINING THE SAME, AND APPLICATION THEREOF

(71) Applicant: Industrial Technology Research Institute, Hsinchu (TW)

(72) Inventors: Chieh-Fang CHENG, Taipei City (TW); Nien-Tzu CHOU, Hsinchu City (TW); Ssu-Yuan WU, Hsinchu City (TW)

(73) Assignee: Industrial Technology Research Institute, Hsinchu (TW)

(21) Appl. No.: 15/826,159

(22) Filed: Nov. 29, 2017

Related U.S. Application Data
(60) Provisional application No. 62/427,982, filed on Nov. 30, 2016.

(51) Int. Cl. C07K 14/54; A61P 17/02

(52) U.S. Cl. CPC C07K 14/5412; A61K 38/00; A61P 17/02

(57) ABSTRACT

The present disclosure provides a peptide for promoting cell migration and/or skin wound healing, including an IL-6-derived peptide, which is designed within the region of the sequence of SEQ ID NO. 1 and of which the sequence includes the sequence of SEQ ID NO. 2, in which the IL-6-derived peptide has about 6-50 amino acids.

### CROSS REFERENCE TO RELATED APPLICATION

This application claims the benefit of U.S. Provisional Application No. 62/427,982, filed on Nov. 30, 2016, the entirety of which is incorporated by reference herein.

### INCORPORATION BY REFERENCE OF SEQUENCE LISTING

A sequence listing submitted as a text file via EFS-Web is incorporated herein by reference. The text file containing the sequence listing is named “0965-A225242-US_Seq_Listing.txt”; its date of creation is Feb. 14, 2018; and its size is 3,390 bytes.

### TECHNICAL FIELD

The technical field relates to a peptide for promoting cell migration and/or skin wound healing, a pharmaceutical composition containing the same, and the use thereof.

### BACKGROUND

The wound healing process can be divided into three phases: inflammation, proliferation, and maturation. Chronic inflammation will delay wound healing, and it is known that TNF-α (tumor necrosis factor α) plays an important role in the induction of chronic inflammation and the delay of wound healing. In contrast, it is known that MCP-1 (monocyte chemoattractant protein-1) is capable of accelerating wound healing of diabetics, and skin cell migration plays a key role in the proliferation phase of the wound healing process.

However, current drugs are generally designed to aim at a single phase of wound healing, such as the inflammation phase or the proliferation phase. Furthermore, at present, it is known that growth factors used for wound healing may have the risk of causing inflammation and even result in the generation of a tumor. In addition, peptide fragments which are too long are sensitive to proteases and storage conditions. Therefore, at present, the development of a wound healing drug which can aim at these two phases at the same time is needed, and the drug not only can promote secretion of MCP-1 which accelerates wound healing but also does not stimulate secretion of TNF-α which induces chronic inflammation and delays wound healing.

### SUMMARY

The present disclosure provides a peptide for promoting cell migration and/or skin wound healing, comprising: an IL-6-derived peptide, which is designed within the region of the sequence of SEQ ID NO. 1 and of which the sequence comprises the sequence of SEQ ID NO. 2, in which the IL-6-derived peptide has about 6-50 amino acids.

The present disclosure also provides a pharmaceutical composition for promoting cell migration and/or skin wound healing, comprising: an IL-6-derived peptide, which is designed within the region of the sequence SEQ ID NO. 1 and of which the sequence comprises the sequence of SEQ ID NO. 2, in which the IL-6-derived peptide has about 6-50 amino acids; and a pharmaceutically acceptable carrier or salt.

The present disclosure further provides a method for promoting cell migration and/or skin wound healing, comprising: administering an effective amount of the pharmaceutical composition for promoting cell migration and/or skin wound healing mentioned above to a subject in need thereof to promote cell migration and/or skin wound healing.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1A shows the effects of a peptide of one embodiment of the present disclosure (Peptide 6) on TNF-α secretion of macrophages of Donor 1;

FIG. 1B shows the effects of a peptide of one embodiment of the present disclosure (Peptide 6) on TNF-α secretion of macrophages of Donor 2;

FIG. 1C shows the effects of a peptide of one embodiment of the present disclosure (Peptide 6) on TNF-α secretion of macrophages of Donor 3;

FIG. 2A shows the effects of a peptide of one embodiment of the present disclosure (Peptide 6) on MCP-1 secretion of human vascular endothelial cell line HUVECs;

FIG. 2B shows the effects of a peptide of one embodiment of the present disclosure (Peptide 6) on MCP-1 secretion of macrophages of Donor 1;

FIG. 2C shows the effects of a peptide of one embodiment of the present disclosure (Peptide 6) on MCP-1 secretion of macrophages of Donor 2;

FIG. 2D shows the effects of a peptide of one embodiment of the present disclosure (Peptide 6) on MCP-1 secretion of macrophages of Donor 3;

FIG. 3A and FIG. 3B show the effects of a peptide of one embodiment of the present disclosure (Peptide 6) on cell migration of human keratinocyte cell line HaCaT. Compared to the control group: *: p<0.05.

FIG. 4A shows the photographs of wounds of mice respectively treated with phosphate buffered saline (PBS), platelet-derived growth factor (PDGF) and a peptide of the present disclosure, Peptide 6, at different time points (Day 0, Day 5, Day 7, Day 10 and Day 12);

FIG. 4B is the quantification result of FIG. 4A and shows the effects of phosphate buffered saline, platelet-derived growth factor and the peptide of the present disclosure, Peptide 6, on wound healing in mice as time increases;

FIG. 5 shows the sequence of different isoforms (ITRI-1, ITRI-2, ITRI-3, ITRI-4, ITRI-5, ITRI-6 and ITRI-7) of the peptide of the present disclosure, Peptide 6, and the D-form amino acids contained in the different isoforms;

FIG. 6 shows the effects of a peptide of one embodiment of the present disclosure (Peptide 6), different isoforms of Peptide 6 (ITRI-1, ITRI-2, ITRI-3, ITRI-4, ITRI-5, ITRI-6 and ITRI-7) and TGF-α (10 ng/ml) on cell migration of human keratinocyte cell line HaCaT. Compared to the control group: *: p<0.05; **: p<0.01; ***: p<0.001;

FIG. 7 shows the effects of IL-6 at different concentrations (100 ng/ml and 50 ng/ml), the peptide of the present disclosure, Peptide 6 (the sequence thereof is SEQ ID NO. 6), at different concentrations (250 μg/ml, 125 μg/ml, 50 μg/ml and 50 ng/ml) and an isoform of Peptide 6, ITRI-2, at different concentrations (250 μg/ml, 125 μg/ml, 50 μg/ml and 50 ng/ml) on TNF-α secretion of macrophages of Donor 4;

FIG. 8 shows the effects of IL-6 at different concentrations (100 ng/ml and 50 ng/ml), the peptide of the present disclosure, Peptide 6 (the sequence thereof is SEQ ID NO. 6), at different concentrations (250 μg/ml, 125 μg/ml, 50 μg/ml and 50 ng/ml) and an isoform of Peptide 6, ITRI-2, at different concentrations (250 μg/ml, 125 μg/ml, 50 μg/ml and 50 ng/ml) on MCP-1 secretion of macrophages of Donor 4;

FIG. 9 shows the effects of the peptide of the present disclosure, Peptide 6 (the sequence thereof is SEQ ID NO. 6), at different concentrations (1.5 mg/ml, 1 mg/ml and 0.5 mg/ml) and the isoform of Peptide 6, ITRI-2, at different concentrations (1.5 mg/ml, 1 mg/ml and 0.5 mg/ml), IL-6 at different concentrations (1000 ng/ml and 100 ng/ml) and TGF-α (10 ng/ml) on MMP9 secretion of human keratinocyte cell line HaCaT;

FIG. 10 shows sequence alignment for Peptide 6 and different short form peptides thereof;

FIG. 11 shows the effects of the peptide of the present disclosure, Peptide 6 (the sequence thereof is SEQ ID NO. 6) (1000 μg/ml) and different short form peptides of Peptide 6 (peptide 6M14 (1000 μg/ml) and peptide 6L21 (1400 μg/ml)) and TGF-α (10 ng/ml) on cell migration of human keratinocyte cell line HaCaT (control group: HaCaT cells not treated);

FIG. 12 shows the effects of the peptide of the present disclosure, Peptide 6 (the sequence thereof is SEQ ID NO. 6) (50 μg/ml), a short form peptide of Peptide 6, 6M14 (50 μg/ml), and IL-6 (200 ng/ml) on TNF-α secretion of macrophages of Donor 5;

FIG. 13 shows the effects of the peptide of the present disclosure, Peptide 6 (the sequence thereof is SEQ ID NO. 6) (50 μg/ml), a short form peptide of Peptide 6, 6M14 (50 μg/ml), and IL-6 (200 ng/ml) on MCP-1 secretion of macrophages of Donor 5.

### DETAILED DESCRIPTION

In the following detailed description, for the purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present disclosure provides a peptide for promoting cell migration and/or skin wound healing. The peptide for promoting cell migration and/or skin wound healing mentioned above may comprise, but is not limited to, an IL-6 (Interleukin 6)-derived peptide. IL-6, which is a kind of interleukin, plays the role of both a pro-inflammatory cytokine and an anti-inflammatory myokine at the same time, and the mature form of IL-6 has the sequence of SEQ ID NO. 1.

The peptide for promoting cell migration and/or skin wound healing mentioned above not only can promote secretion of MCP-1 which accelerates wound healing without stimulating secretion of TNF-α which induces chronic inflammation and delays wound healing, but also can promote cell migration to converge, heal and trim the wounds.

In the peptide for promoting cell migration and/or skin wound healing of the present disclosure mentioned above, the foregoing IL-6-derived peptide may be designed within the region of the sequence of SEQ ID NO. 1. The sequence of the foregoing IL-6-derived peptide may comprise the sequence of SEQ ID NO. 2, but it is not limited thereto. Moreover, the foregoing IL-6-derived peptide may have about 6-50 amino acids, such as about 14-48 amino acids, but it is not limited thereto. For example, the foregoing IL-6-derived peptide may have 6, 14, 21 or 28 amino acids.

In one embodiment, in the peptide for promoting cell migration and/or skin wound healing of the present disclosure, the sequence of the foregoing IL-6-derived peptide may be the sequence of SEQ ID NO. 2.

Moreover, in one embodiment of the peptide for promoting cell migration and/or skin wound healing of the present disclosure, the sequence of the foregoing IL-6-derived peptide may comprise the sequence of SEQ ID NO. 3, but it is not limited thereto. In another embodiment of the peptide for promoting cell migration and/or skin wound healing of the present disclosure, the sequence of the foregoing IL-6-derived peptide may comprise the sequence of SEQ ID NO. 4, but it is not limited thereto. Furthermore, in another embodiment of the peptide for promoting cell migration and/or skin wound healing of the present disclosure, the sequence of the foregoing IL-6-derived peptide may comprise the sequence of SEQ ID NO. 5, but it is not limited thereto. In further another embodiment of the peptide for promoting cell migration and/or skin wound healing of the present disclosure, the sequence of the foregoing IL-6-derived peptide may comprise, but is not limited to, the sequence of SEQ ID NO. 6.

In addition, in one embodiment of the peptide for promoting cell migration and/or skin wound healing of the present disclosure, the foregoing IL-6-derived peptide may have about 14-28 amino acids. In this embodiment, the sequence of the foregoing IL-6-derived peptide may be the sequence of SEQ ID NO. 3, the sequence of SEQ ID NO. 4, the sequence of SEQ ID NO. 5 or the sequence of SEQ ID NO. 6.

Furthermore, in another embodiment, in the peptide for promoting cell migration and/or skin wound healing of the present disclosure, the foregoing IL-6-derived peptide may have at least one D-form amino acid.

In the IL-6-derived peptide, in one embodiment, the positions of the D-form amino acids may comprise, but are not limited to (a) the first amino acid in front of the at least one metalloproteinase cleavage site of the IL-6-derived peptide, (b) the first amino acid in front of the at least one serine proteases cleavage site of the IL-6-derived peptide, (c) the first amino acid in front of the at least one cleavage site for another enzyme which exists in a wound of the IL-6-derived peptide, or a combination thereof.

The first amino acid in front of the at least one metalloproteinase cleavage site of the IL-6-derived peptide mentioned above may comprise the amino acid(s) of the foregoing IL-6-derived peptide corresponding to position 113 of the sequence of SEQ ID NO. 1, corresponding to position 115 of the sequence of SEQ ID NO. 1, corresponding to position 120 of the sequence of SEQ ID NO. 1, corresponding to position 124 of the sequence of SEQ ID NO. 1, or a combination thereof, but it is not limited thereto.

The first amino acid in front of the at least one serine proteases cleavage site of the IL-6-derived peptide mentioned above may comprise the amino acid(s) of the foregoing IL-6-derived peptide corresponding to position 116 of the sequence of SEQ ID NO. 1, corresponding to position 122 of the sequence of SEQ ID NO. 1, or a combination thereof, but it is not limited thereto.

The first amino acid in front of the least one cleavage site for another enzyme which exists in a wound of the IL-6-derived peptide mentioned above may comprise the amino acid(s) of the foregoing IL-6-derived peptide corresponding to position 114 of the sequence of SEQ ID NO. 1, but it is not limited thereto.

Furthermore, in the embodiment in which the foregoing IL-6-derived peptide may have at least one D-form amino acid, the sequence of the foregoing IL-6-derived peptide may comprise the sequence of SEQ ID NO. 6, but it is not limited thereto. In addition, in this embodiment, in the condition in which the sequence of the foregoing IL-6-derived peptide may comprise the sequence of SEQ ID NO. 6, the D-form amino acid mentioned above may comprise, but is not limited to, (a) alanine at position 12 of the sequence of SEQ ID NO. 6, (b) valine at position 13 of the sequence of SEQ ID NO. 6, (c) glutamine at position 14 of the sequence of SEQ ID NO. 6, (d) methionine at position 15 of the sequence of SEQ ID NO. 6, (e) valine at position 19 of the sequence of SEQ ID NO. 6, (0 isoleucine at position 21 of the sequence of SEQ ID NO. 6, (g) phenylalanine at position 23 of the sequence of SEQ ID NO. 6, or any combination thereof.

In one embodiment, the foregoing IL-6-derived peptide may have at least one D-form amino acid, and the sequence of the foregoing IL-6-derived peptide may be SEQ ID NO. 6.

In one specific embodiment, the foregoing IL-6-derived peptide may have at least one D-form amino acid, and the sequence of the foregoing IL-6-derived peptide may be SEQ ID NO. 6, in which the D-form amino acid may be alanine at position 12 of the sequence of SEQ ID NO. 6. Moreover, in one specific embodiment, the foregoing IL-6-derived peptide may have at least one D-form amino acid, and the sequence of the foregoing IL-6-derived peptide may be SEQ ID NO. 6, in which the D-form amino acid may be valine at position 13 of the sequence of SEQ ID NO. 6. In another specific embodiment, the foregoing IL-6-derived peptide may have at least one D-form amino acid, and the sequence of the foregoing IL-6-derived peptide may be SEQ ID NO. 6, in which the D-form amino acid may be glutamine at position 14 of the sequence of SEQ ID NO. 6. Furthermore, in another specific embodiment, the foregoing IL-6-derived peptide may have at least one D-form amino acid, and the sequence of the foregoing IL-6-derived peptide may be SEQ ID NO. 6, in which the D-form amino acid may be methionine at position 15 of the sequence of SEQ ID NO. 6. In another specific embodiment, the foregoing IL-6-derived peptide may have at least one D-form amino acid, and the sequence of the foregoing IL-6-derived peptide may be SEQ ID NO. 6, in which the D-form amino acid may be valine at position 19 of the sequence of SEQ ID NO. 6. In another specific embodiment, the foregoing IL-6-derived peptide may have at least one D-form amino acid, and the sequence of the foregoing IL-6-derived peptide may be SEQ ID NO. 6, in which the D-form amino acid may be isoleucine at position 21 of the sequence of SEQ ID NO. 6. In addition, in another specific embodiment, the foregoing IL-6-derived peptide may have at least one D-form amino acid, and the sequence of the foregoing IL-6-derived peptide may be SEQ ID NO. 6, in which the D-form amino acid may be phenylalanine at position 23 of the sequence of SEQ ID NO. 6.

The present disclosure also provides a pharmaceutical composition for promoting cell migration and/or skin wound healing.

The pharmaceutical composition for promoting cell migration and/or skin wound healing of the present disclosure may comprise, but is not limited to, any above-mentioned IL-6-derived peptide of the peptide for promoting cell migration and/or skin wound healing of the present disclosure, and a pharmaceutically acceptable carrier or salt.

In the any above-mentioned pharmaceutical composition for promoting cell migration and/or skin wound healing, the pharmaceutically acceptable carrier mentioned above may comprise, but is not limited to, a solvent, a dispersion medium, a coating, an antibacterial and antifungal agent, or an isotonic and absorption delaying agent, etc., which is suitable for pharmaceutical administration. The pharmaceutical composition can be formulated into dosage forms for different administration routes utilizing conventional methods.

The pharmaceutically acceptable salt mentioned above may include, but is not limited to, salts including inorganic cation, such as alkali metal salts such as sodium salt, potassium salt or amine salt, such as alkaline-earth metal salt such as magnesium salt or calcium salt, such as the salt containing bivalent or quadrivalent cation such as zinc salt, aluminum salt or zirconium salt. In addition, the pharmaceutically acceptable salt may also be organic salt, such as dicyclohexylamine salt, methyl-D-glucamine, and amino acid salt such as arginine, lysine, histidine, or glutamine.

The pharmaceutical composition of the present disclosure may be administered orally, parenterally, by an inhalation spray, or via an implanted reservoir. The parenteral methods may comprise smearing affected regions, subcutaneous, intracutaneous, intravenous, intramuscular, intra-articular, intra-arterial, intrasynovial, intrasternal, intrathecal, and intralesional injection, as well as infusion techniques. Forms of topical composition for smearing may comprise ointments, creams, solutions, gels, etc. but they are not limited thereto.

The forms of oral composition may include, but is not limited to, tablets, capsules, emulsions, aqueous suspensions, dispersions and solutions.

Furthermore, the present disclosure further provides a method for promoting cell migration and/or skin wound healing. The method may include, but is not limited to, administering an effective amount of any above-mentioned pharmaceutical composition for promoting cell migration and/or skin wound healing of the present disclosure to a subject in need thereof to promote cell migration and/or skin wound healing.

The above-mentioned pharmaceutical composition for promoting cell migration and/or skin wound healing of the present disclosure not only can promote secretion of MCP-1 which accelerates wound healing without stimulating secretion of TNF-α which induces chronic inflammation and delays wound healing, but also can promote cell migration to converge, heal and trim the wounds. Moreover, since the pharmaceutical composition for promoting cell migration and/or skin wound healing of the present disclosure can promote secretion of MCP-1 which accelerates wound healing while does not stimulate secretion of TNF-α which induces chronic inflammation and delays wound healing, it is applicable to inflammatory phase of wound healing. Furthermore, since the pharmaceutical composition for promoting cell migration and/or skin wound healing of the present disclosure is capable of promoting cell migration, it is also applicable to proliferation phase of wound healing at the same time.

In any above-mentioned method for promoting cell migration and/or skin wound healing of the present disclosure, the subject may include a cell, a tissue, an animal, etc. Examples of the tissue may include, but are not limited to, a skin tissue. Moreover, the animal mentioned above may comprise a mammal, but it is not limited thereto. Examples of the mammal may include, but are not limited to, a human, an orangutan, a monkey, a horse, a donkey, a dog, a cat, a rabbit, a guinea pig, a rat, and a mouse. In one embodiment, in any above-mentioned method for promoting cell migration and/or skin wound healing of the present disclosure, the subject is a human.

### EXAMPLES

A. Material

1. Reagents, Kits and Equipment

1.1 Ficoll Hypaque: Histopaque®-1077 (Sigma Cat#10771);

1.2 Human MACS CD14 isolation kit (MACS Cat#130-050-201);

1.3 RPMI 1640 Medium (Gibco BRL Cat#11875), supplemented with:
10% heat-deactivated (56° C., 45 minutes) fetal bovine serum (FBS) (Invitrogen, Carlsbad, Calif., USA);
100× penicillin-streptomycin solution diluted with a ratio of 1:100 (1% P/S) (Invitrogen, Carlsbad, Calif., USA);
30 ng/ml M-CSF (Peprotech Cat# AF-300-03);

1.4 Human TNF-α ELISA Kit (R&D Systems Cat# DY210)

1.5 Human MCP-1 ELISA Kit (R&D Systems Cat# DY279)

1.6 6-Well Culturing Plate (Costar Cat #3516);

1.7 Respective Aseptic Packages of 5 mL, 10 mL and 25 mL serological pipets (Costar, Cambridge, Mass., USA)

1.8 Electric Pipet-Aid (Gilson, UK).

2. Cells

2.1 Human Monocyte-Derived Macrophages

2.1.1 Source: Obtained from the blood of donors provided by the Taiwan Blood Services Foundation.

2.1.2 Medium: RPMI 1640 medium supplemented with 10% fetal bovine serum, 100 U/ml penicillin, 100 U/ml streptomycin, and 30 ng/ml M-CSF

2.2 Human Keratinocyte Cells

2.2.1 Cell Line: HaCaT

2.2.2 Source: HaCaT purchased from Bioresource Collection and Research Center (BCRC) of Food Industry Research and Development Institute (FIRDI).

2.3 Human Vein Endothelial Cells

2.3.1 Cell Line: Human umbilical vein endothelial cell (HUVEC)

2.3.2 Source: HUVEC purchased from American Type Culture Collection (ATCC) (https://www.atcc.org/products/all/CRL-1730.aspx).

2.2.3 Medium: DMEM supplemented with 10% fetal bovine serum

3. Experimental Animals

3.1 Mice (Nude Mice)

3.2 Age: 8 to 10 week-old

3.3 Source: purchasing from BioLASCO Taiwan Co., Ltd

4. Peptides

The peptides of the present disclosure were synthesized by Genescript.

B. Method

1. Obtainment of Human Monocyte-Derived Macrophages

1.1 Obtainment of Peripheral Blood Mononuclear Cells (PBMCs)

Peripheral blood mononuclear cells were isolated from leukocytes in the blood of healthy donors by Ficoll Hypaque.

1.2 Isolation of Monocytes for Generating Macrophages

Monocytes were isolated from the peripheral blood mononuclear cells by using Human MACS CD14 Isolation Kit.

1.3 Formation and Culturing of Macrophages

Monocytes (2×10⁶ cells/mL) were seeded in a 6-well plate containing 2 mL fresh complete medium and cultured at 5% CO₂, 37° C. for 6 days. The medium was changed every 2-3 days to make the monocytes differentiate to macrophages.

2. Detection of TNF-α Secretion of Monocyte-Derived Macrophages Stimulated with Peptides

2.1 Stimulation of Macrophages with Peptides

Peptides were added to respective wells of a culturing plate to stimulate macrophages for 48 hours. Macrophages not stimulated were used as a blank group.

2.2 Detection of TNF-α Secreted by Macrophages by ELISA Analysis

2.2.1 TNF-α secreted from macrophages to the supernatant of the culture was detected by a human TNF-α kit.

2.2.2 Absorbance of the supernatant of the culture at 450-540 nm was detected by a SpectraMax M5 microplate reader. Detection limitation of this ELISA kit to TNF-α is about 15.6 μg/ml.

3. Detections of MCP-1 Secretion of Human Umbilical Vein Endothelial Cells HUVECs and Human Monocyte-Derived Macrophages Stimulated with Peptides

3.1 Stimulation of Human Umbilical Vein Endothelial Cells and Macrophages with Peptides

Peptides were added to respective wells of a culturing plate to stimulate HUVECs for 24-48 hours, or to stimulate macrophages for 48 hours. In the experiment for macrophages, macrophages not stimulated were used as a blank group.

3.2 Detections of MCP-1 Secreted by HUVECs and Macrophages by ELISA Analysis

3.2.1 MCP-1 respectively secreted from HUVECs and macrophages to the supernatant of the culture was detected by a human TNF-α kit (R&D systems, Human TNF-alpha DuoSet ELISA (DY210)).

3.2.2 Absorbance of the supernatant of the culture at 450-540 nm was detected by a SpectraMax M5 microplate reader. Detection limitation of this ELISA kit to MCP-1 is about 15.6 μg/ml.

4. Cell Migration Analysis of Human Keratinocyte Cell Line HaCaT Stimulated with Peptides

4.1 Each well of a 12-well culturing plate was coated with 100 μg/ml of collagen (in 0.02N acetic acid) (0.5 ml/well).

4.2 The culturing plate was placed at 37° C. for 1 hour, and then was washed with Dulbecco's phosphate buffered saline (DPBS) one time.

4.3 HaCaT cells were seeded in the culturing plate with DMEM containing serum (5×10⁵ cells/well), and the final serum concentration for each well was 10%.

4.4 The cells were cultured in a condition of 5% CO₂, 37° C.

4.5 The medium was replaced with DMEM containing 0.6% serum for serum starvation and culture the cells overnight.

4.6 The bottom of the well of the culturing plate was scraped with 1000 μl tips, and the culturing plate was washed with Dulbecco's phosphate buffered saline twice.

4.7 The cells and peptides were cultured in DMEM containing 0.6% serum. HaCaT cells not stimulated with peptides or growth factors were used as a control group.

4.8 Photographs were taken at 0 hour and 18 hour.

4.9. The photographs were analyzed by Celllmage Comp software, and the migration distances for cells were calculated.

5. Detection of MMP9 Secretion of Human Keratinocyte Cell Line HaCaT Stimulated with Peptides

5.1 Stimulation of HaCaT Cells with Peptides

Peptides were added to respective wells of a culturing plate to stimulate HaCaT cells for 48 hours. HaCaT cells not stimulated were used as a blank group.

5.2 Detection of MMP9 Secreted by HaCaT Cells by ELISA Analysis

5.2.1 MMP9 secreted from HaCaT cells to the supernatant of the culture was detected by a MMP9 kit (R&D systems, Human MMP-9 DuoSet ELISA (DY911)).

5.2.2 Absorbance of the supernatant of the culture at 450-540 nm was detected by a SpectraMax M5 microplate reader. Detection limitation of this ELISA kit to MMP9 is about 31.3 μg/ml.

6. Wound Healing Analysis for Wound of Mouse Treated with Peptide

6.1 A full-thickness excision wound (1×1 cm²) was made on the mid-backs of 8-10 week-old mice by scissors.

6.2 The wound was topically covered by 100 μl of 10% carboxymethyl cellulose (CMC) gel with peptide or platelet-derived growth factor (PDGF) or without peptide or platelet-derived growth factor, and then covered by a band-aid and Coban (without using a specific brand; purchased from a common pharmacy) to prevent from drying. Peptide and platelet-derived growth factor was provided one time on Day 0, and photographs of the wounds were taken on Day 0, Day 5, Day 10 and Day 12. The mice of the control group were only treated with excipient (5% carboxymethyl cellulose gel).

### Example 1

Analysis of the Effects of the Peptides of the Present Disclosure

1. Effects of Peptides of the Present Disclosure on TNF-α Secretion of Human Monocyte-Derived Macrophages

TNF-α is an important cytokine during inflammatory phase of wound healing, and it is known that TNF-α will induce chronic inflammation and delay wound healing. Therefore, during wound healing, it is generally undesirable that TNF-α is over-secreted. In this experiment, the effects of the peptides of the present disclosure on TNF-α secretion of human monocyte-derived macrophages were determined.

The peptide adopted in this experiment was Peptide 6 and the sequence thereof was SEQ ID NO. 6 that corresponds to amino acids of positions 102 to 129 of mature form IL-6 (SEQ ID NO. 1).

Macrophages obtained from the blood of different donors were stimulated with IL-6 (50 ng/ml) and the peptide of the present disclosure, Peptide 6 (the sequence of which was SEQ ID NO. 6), at different concentrations (Donor 1: 660 μg/ml; Donor 2: 1000 μg/ml, 500 μg/ml and 250 μg/ml; Donor 3: 500 μg/ml, 125 μg/ml, 50 ng/ml and 6 ng/ml), and TNF-α secretion of macrophages was detected (macrophages not stimulated were used as a blank group).

The experimental method is the same as above-mentioned item “2. Detection of TNF-α secretion of monocyte-derived macrophages stimulated with peptides” of “B. Method” paragraph.

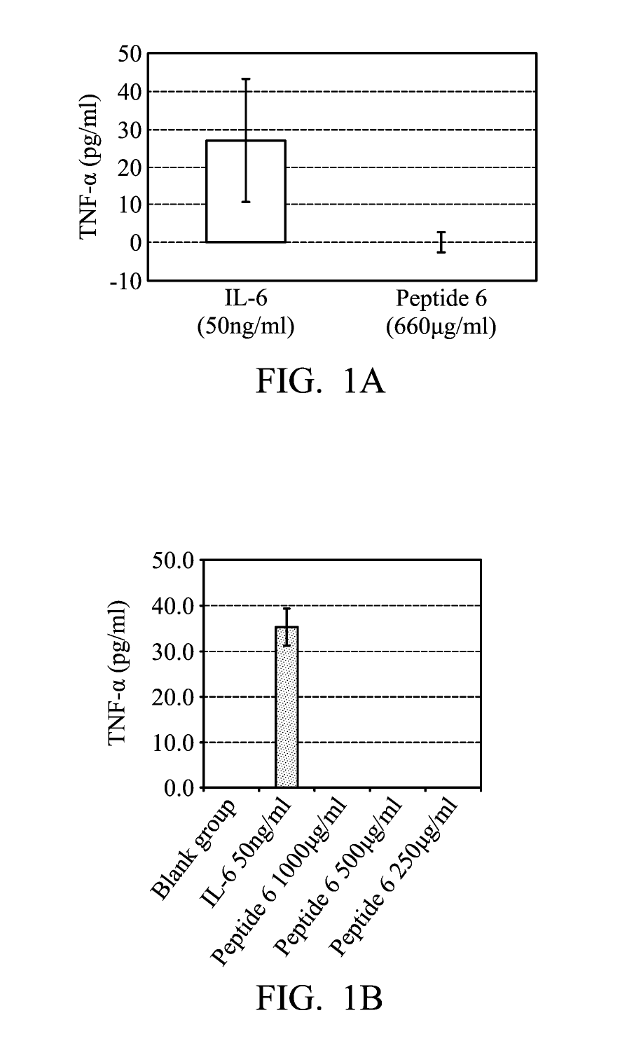

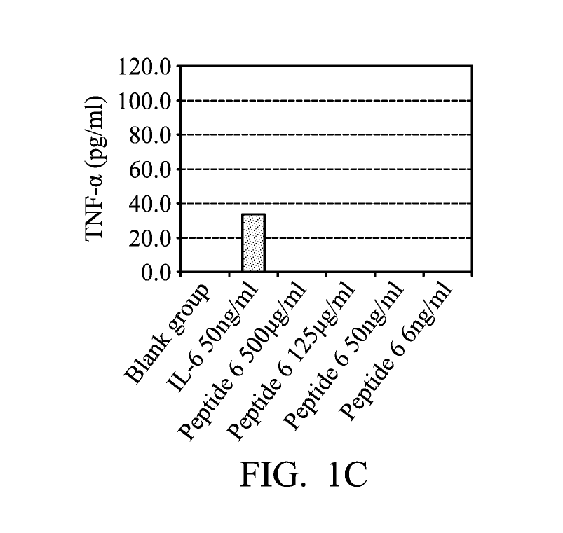

The results of stimulating macrophages of different donors and determining TNF-α secretion thereof are shown in FIG. 1A, FIG. 1B and FIG. 1C, respectively.

As illustrated in FIG. 1A, FIG. 1B and FIG. 1C, the peptide of the present disclosure, Peptide 6, does not induce macrophages to secrete TNF-α, even at a test concentration as high as 1000 μg/ml. In contrast, IL-6, even if at a low concentration such as 50 ng/ml, can induce macrophages to secrete TNF-α.

2. Effects of Peptides of the Present Disclosure on MCP-1 Secretion of Human Umbilical Vein Endothelial Cells (HUVEC) and Human Monocyte-Derived Macrophages

MCP-1 is also an important cytokine during inflammatory phase of wound healing, and it is known that MCP-1 is capable of accelerating wound healing of diabetics. In this experiment, the effects of the peptides of the present disclosure on MCP-1 secretion of human umbilical vein endothelial cells (HUVEC) and human monocyte-derived macrophages were determined.

Human vascular endothelial cell line HUVECs were stimulated with IL-6 at different concentrations (200 ng/ml and 50 ng/ml) and the peptide of the present disclosure, Peptide 6 (the sequence of which was SEQ ID NO. 6), at different concentrations (660 μg/ml and 165 μg/ml) and MCP-1 secretion of HUVECs was detected.

Macrophages obtained from the blood of different donors were stimulated with IL-6 at different concentrations (Donor 1: 50 ng/ml; Donor 2 and Donor 3: 200 ng/ml, 50 ng/ml and 5 ng/ml) and the peptide of the present disclosure, Peptide 6 (the sequence of which was SEQ ID NO. 6), at different concentrations (Donor 1: 660 μg/ml; Donor 2: 1000 μg/ml, 500 μg/ml and 250 μg/ml; Donor 3: 500 μg/ml and 125 μg/ml) and MCP-1 secretion of macrophages was detected (macrophages not stimulated were used as a blank group).

The experimental method is the same as above-mentioned item “3. Detections of MCP-1 secretion of human vein endothelial cells HUVEC and human monocyte-derived macrophages stimulated with peptides” of “B. Method” paragraph.

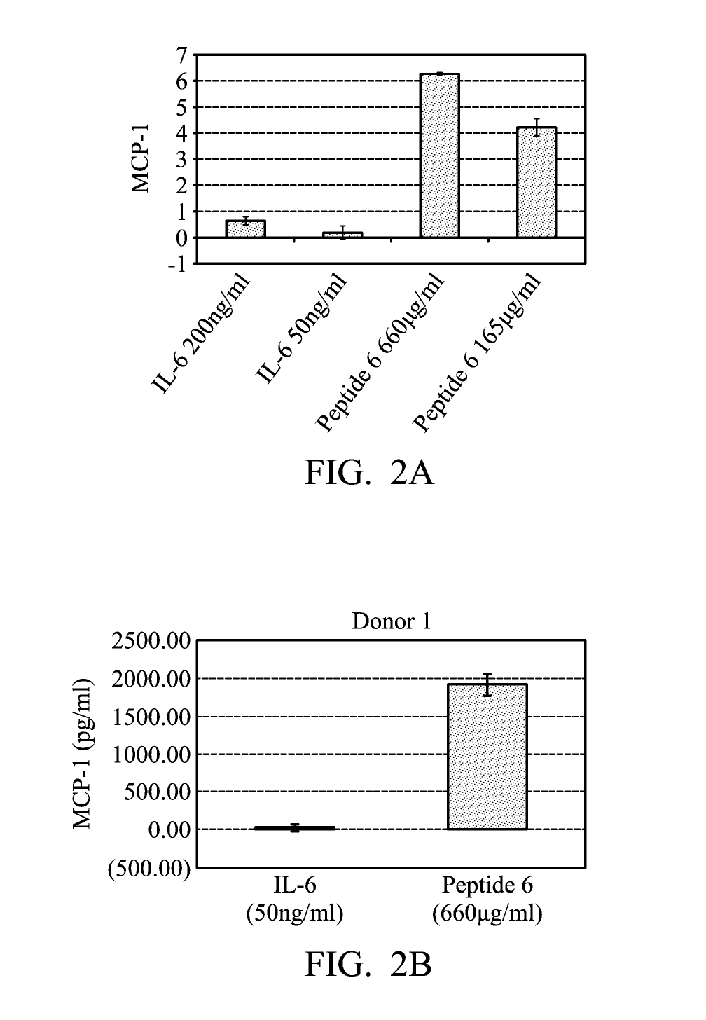

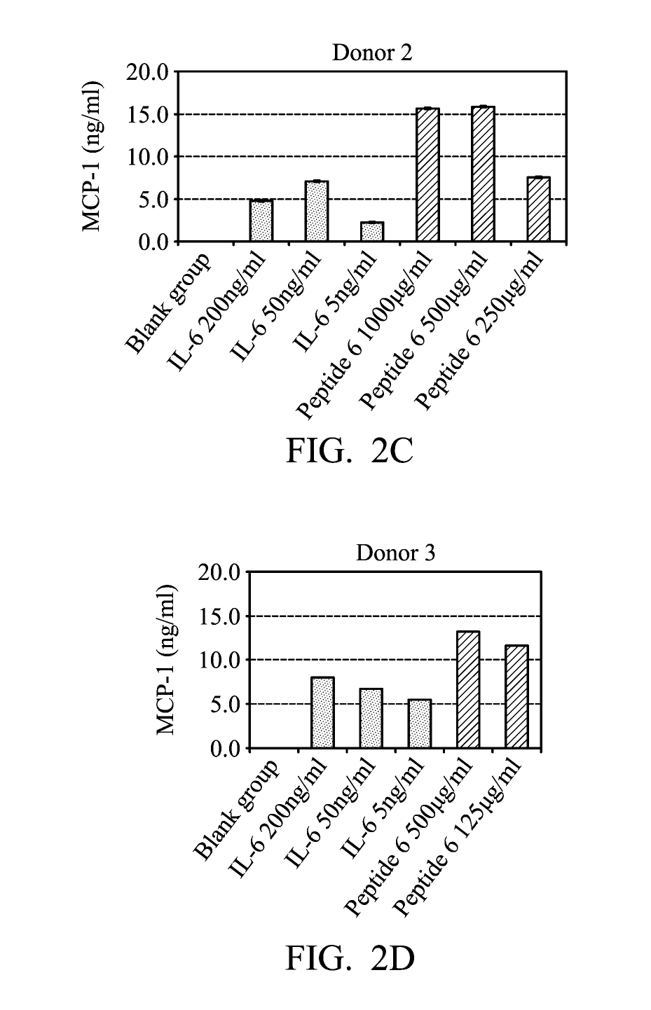

The MCP-1 determining results for HUVECs are shown in FIG. 2A, and the MCP-1 determining results for macrophages of different donors are shown in FIG. 2B, FIG. 2C and FIG. 2D, respectively.

As illustrated in FIG. 2A, FIG. 2B, FIG. 2C and FIG. 2D, the peptide of the present disclosure, Peptide 6, could induce human vascular endothelial cell line HUVECs or macrophages to secrete a great quantity of MCP-1. In contrast, IL-6 induced human vascular endothelial cells or macrophages to secrete a lower quantity of MCP-1.

3. Effects of Peptides of the Present Disclosure on Cell Migration of Human Keratinocyte Cells

Cell migration is an important step during proliferation phase of wound healing. In this experiment, the effects of the peptides of the present disclosure on cell migration of human keratinocyte cells were determined.

Human keratinocyte cell line HaCaT cells were stimulated with IL-6 at different concentrations (20 ng/ml, 5 ng/ml and 1 ng/ml), the peptide of the present disclosure, Peptide 6 (the sequence of which was SEQ ID NO. 6), at different concentrations (1000 μg/ml, 300 μg/ml, 100 μg/ml and 660 μg/ml), TGF-α (10 ng/ml) and insulin (5 μg/ml), and cell migration level of the HaCaT cells was determined (HaCaT cells not stimulated with a peptide or growth factor were used as a control group).

The experimental method is the same as above-mentioned item “4. Cell migration analysis of human keratinocyte cell line HaCaT stimulated with peptides” of “B. Method” paragraph.

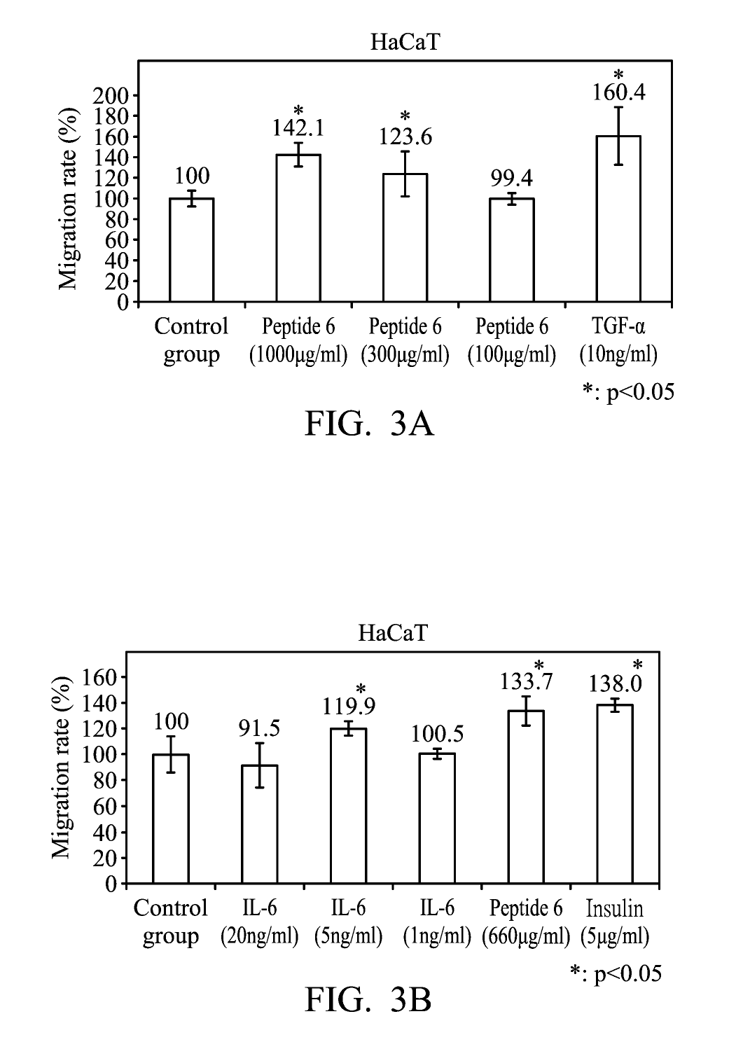

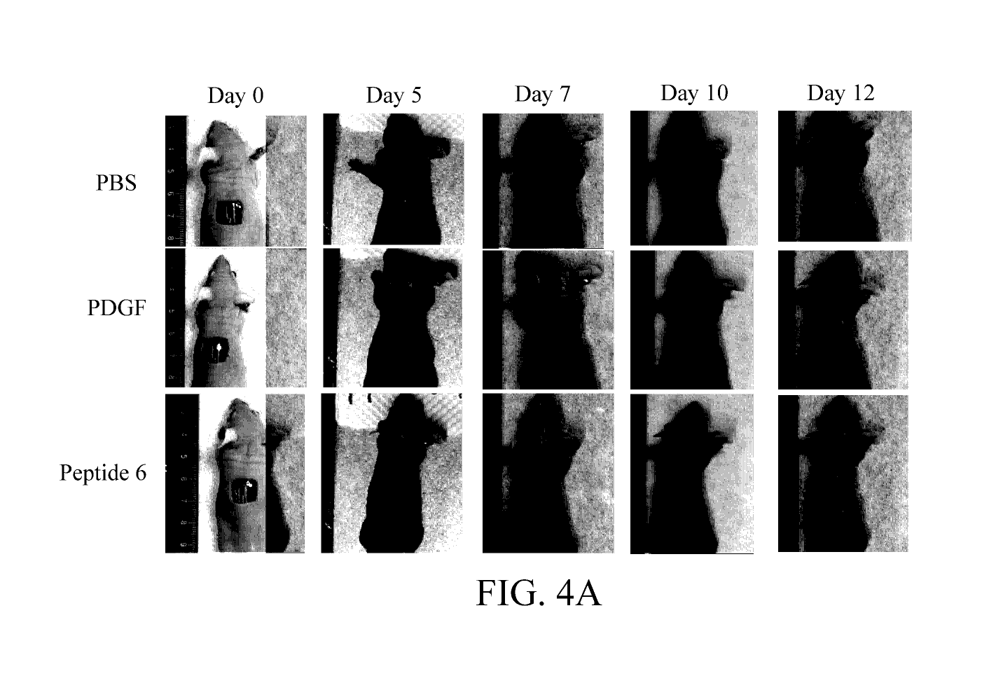

The results are shown in FIG. 3A and FIG. 3B. As illustrated in FIG. 3A and FIG. 3B, when compared to the control group, the peptide of the present disclosure, Peptide 6, can promote cell migration of human keratinocyte cell HaCaT at concentrations of 300 μg/ml, 660 μg/ml and 1000 μg/ml.

4. Effects of Peptides of the Present Disclosure on Wound Healing for Wounds of Mice

Full-thickness excision wounds on the mid-backs of mice were treated with phosphate buffered saline (PBS), platelet-derived growth factor (PDGF) (5 μg) and the peptide of the present disclosure, Peptide 6, (the sequence of which was SEQ ID NO. 6) (1 mg), respectively, and wound healing levels thereof were analyzed (The mice of the control group were only treated with excipient (5% carboxymethyl cellulose gel)).

The experimental method is the same as above-mentioned item “6. Wound healing analysis for wound of mouse treated with peptide” of “B. Method” paragraph.

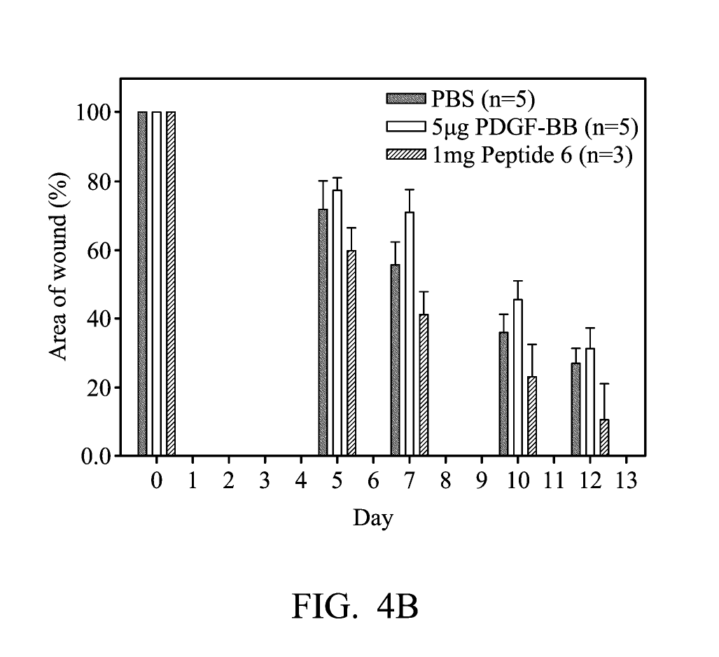

The results are shown in FIG. 4A and FIG. 4B. FIG. 4A shows photographs of wounds of mice respectively treated with phosphate buffered saline, platelet-derived growth factor (PDGF) (5 μg) and the peptide of the present disclosure, Peptide 6 at different time points (Day 0, Day 5, Day 7, Day 10 and Day 12). FIG. 4B is the quantification result for FIG. 4A and shows the effects of phosphate buffered saline, platelet-derived growth factor and the peptide of the present disclosure, Peptide 6, respectively, on wound healing in mice as time increases.

### Example 2

Analysis of the Effects of Isoforms of a Peptide of the Present Disclosure

Cleavage sites for various enzymes which generally exist in a wound of the peptide of the present disclosure, Peptide 6 (the sequence of which was SEQ ID NO. 6), were analyzed.

Cleavage site analysis results show that metalloproteinase cleavage sites locate between position 12 and position 13, between position 14 and position 15, between position 19 and position 20, and between position 23 and position 24 of the sequence of Peptide 6. Serine proteases cleavage sites locate between position 15 and position 16 and between position 21 and position 22 of the sequence of Peptide 6. Cleavage site for another enzyme which exists in a wound locates between position 13 and position 14 of the sequence of Peptide 6.

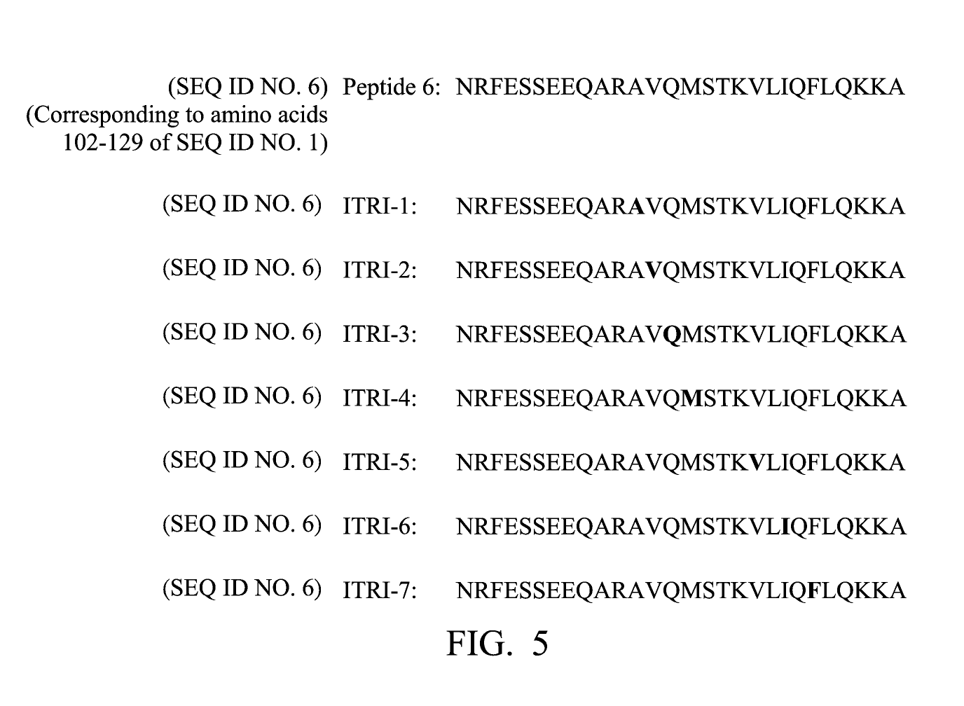

Therefore, based on the information of cleavage sites for respective enzymes, 7 isoform peptides of a peptide of the present disclosure were synthesized, in which the sequences of the isoform peptides were the same as the sequence of Peptide 6 while each of them had a D-form amino acid. The position of the D-form amino acid for each isoform is indicated in FIG. 5 and is described as follows:

ITRI-1: alanine at position 12;

ITRI-2: valine at position 13;

ITRI-3: glutamine at position 14;

ITRI-4: methionine at position 15;

ITRI-5: valine at position 19;

ITRI-6: isoleucine at position 21;

ITRI-7: phenylalanine at position 23.

1. Effects of Isoforms of the Peptide of the Present Disclosure on Cell Migration of Human Keratinocyte Cell

In this experiment, the effects of isoforms of the peptide of the present disclosure on cell migration of human keratinocyte cells were determined.

Human keratinocyte cell line HaCaT cells were stimulated with the peptide of the present disclosure, Peptide 6 (the sequence thereof was SEQ ID NO. 6) (500 μg/ml), different isoforms of Peptide 6 (ITRI-1, ITRI-2, ITRI-3, ITRI-4, ITRI-5, ITRI-6 and ITRI-7) (500 μg/ml) and TGF-α (10 ng/ml), and cell migration levels of the HaCaT cells were determined (HaCaT cells not stimulated with a peptide or growth factor were used as a control group).

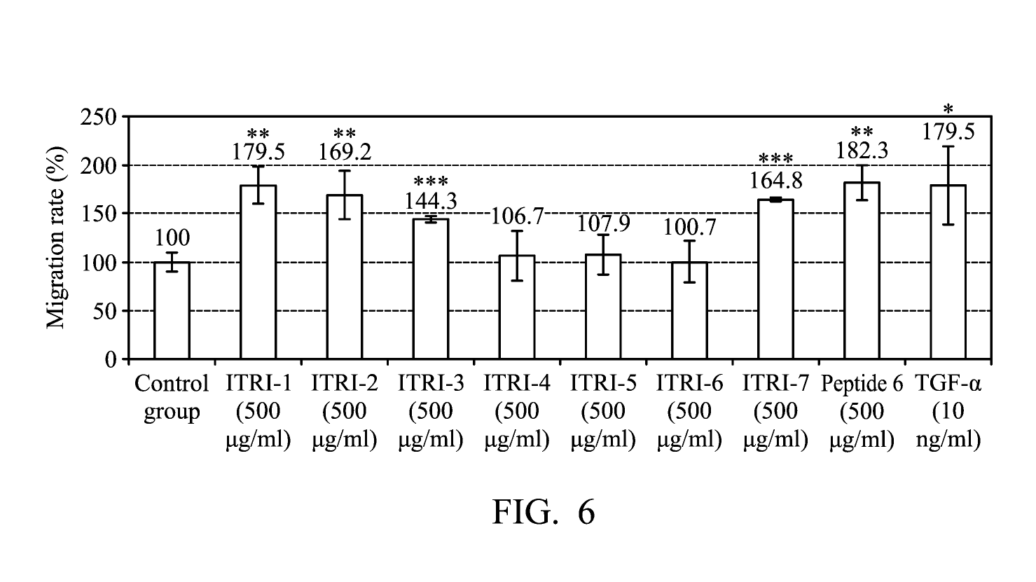

The experimental method is the same as above-mentioned item “4. Cell migration analysis of human keratinocyte cell line HaCaT stimulated with peptides” of “B. Method” paragraph. The results are shown in FIG. 6.

As illustrated in FIG. 6, the different isoforms of Peptide 6 mentioned above all have the effect of promoting cell migration.

2. Effects of Isoforms of the Peptide of the Present Disclosure on TNF-α Secretion of Human Monocyte-Derived Macrophages

In this experiment, the effects of isoforms of the peptide of the present disclosure on TNF-α secretion of human monocyte-derived macrophages were determined.

Macrophages obtained from the blood of Donor 4 were stimulated with IL-6 at different concentrations (100 ng/ml and 50 ng/ml), the peptide of the present disclosure, Peptide 6 (the sequence thereof was SEQ ID NO. 6), at different concentrations (250 μg/ml, 125 μg/ml, 50 μg/ml and 50 ng/ml) and the isoform of the peptide of the present disclosure, Peptide 6, ITRI-2, at different concentrations (250 μg/ml, 125 μg/ml, 50 μg/ml and 50 ng/ml), and TNF-α secretion of macrophages was detected (macrophages not stimulated were used as a blank group).

The experimental method is the same as above-mentioned item “2. Detection of TNF-α secretion of monocyte-derived macrophages stimulated with peptides” of “B. Method” paragraph.

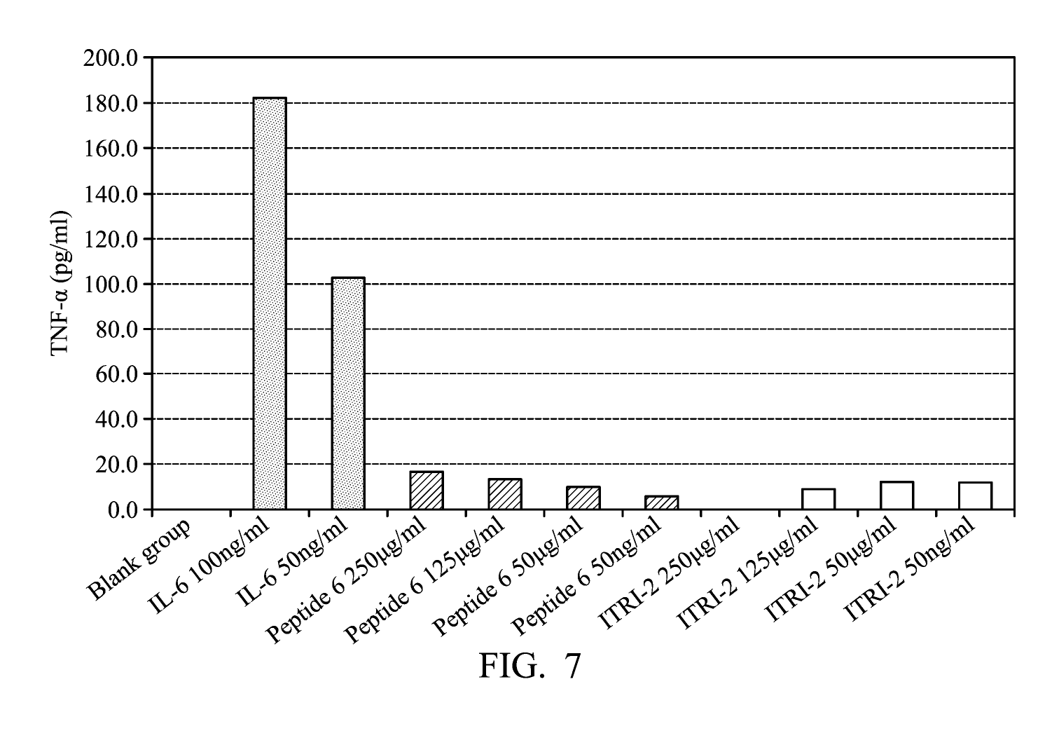

The results of stimulating macrophages of Donor 4 and determining TNF-α secretion thereof are shown in FIG. 7.

As illustrated in FIG. 7, even at a high concentration of 250 μg/ml, the peptide of the present disclosure, Peptide 6, and an isoform thereof ITRI-2 can only induce macrophages to generate a low level of TNF-α compared to IL-6, which even at a low concentration such as 100 ng/ml and 50 ng/ml, can induce macrophages to secrete a great quantity of TNF-α.

3. Effects of Isoforms of the Peptide of the Present Disclosure on MCP-1 Secretion of Human Monocyte-Derived Macrophages

In this experiment, the effects of isoforms of the peptide of the present disclosure on MCP-1 secretion of human monocyte-derived macrophages were determined.

Macrophages obtained from the blood of Donor 4 were stimulated with IL-6 at different concentrations (100 ng/ml and 50 ng/ml), the peptide of the present disclosure, Peptide 6 (the sequence thereof was SEQ ID NO. 6), at different concentrations (250 μg/ml, 125 μg/ml, 50 μg/ml and 50 ng/ml) and an isoform of the peptide of the present disclosure, Peptide 6, ITRI-2, at different concentrations (250 μg/ml, 125 μg/ml, 50 μg/ml and 50 ng/ml), and MCP-1 secretion of macrophages was detected (macrophages not stimulated were used as a blank group).

The experimental method is the same as above-mentioned item “3. Detections of MCP-1 secretion of human umbilical vein endothelial cells (HUVEC) and human monocyte-derived macrophages stimulated with peptides” of “B. Method” paragraph.

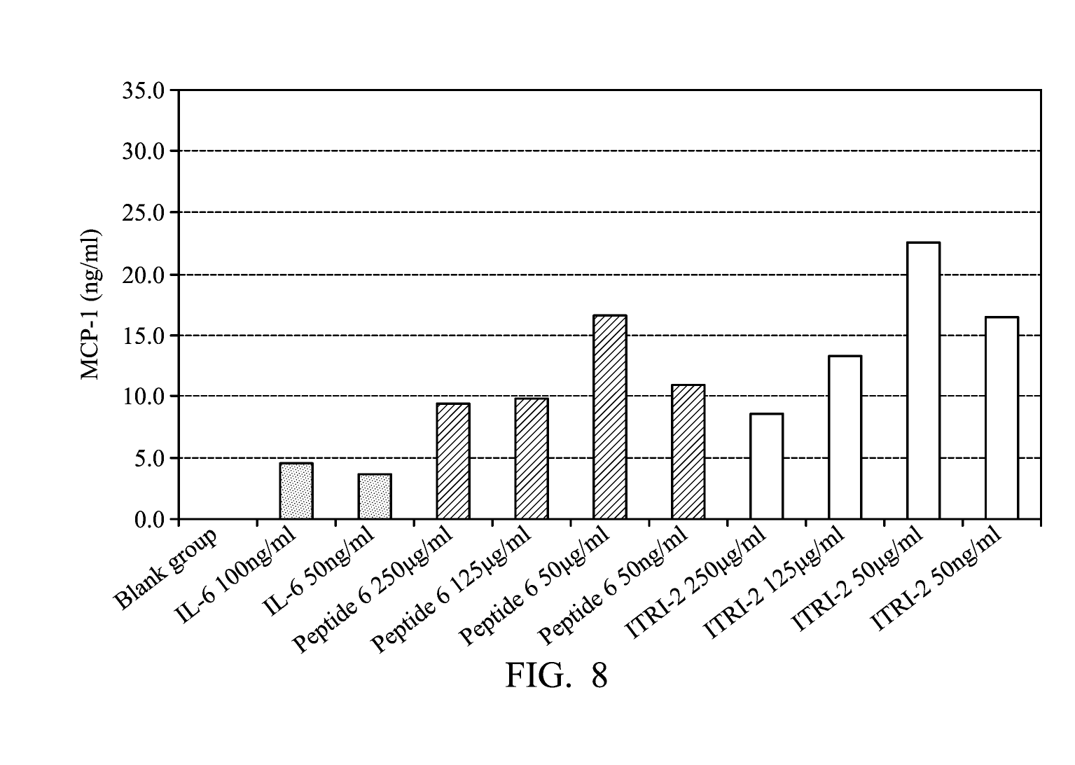

The results of stimulating macrophages of Donor 4 and determining MCP-1 secretion thereof are shown in FIG. 8.

As illustrated in FIG. 8, the peptide of the present disclosure, Peptide 6, and an isoform thereof ITRI-2 are capable of inducing a higher level of MCP-1 secretion than IL-6.

4. Effects of Isoforms of the Peptide of the Present Disclosure on MMP9 Secretion of Human Keratinocyte Cell Line HaCaT

Human keratinocyte cell line HaCaT cells were stimulated with the peptide of the present disclosure, Peptide 6 (the sequence thereof was SEQ ID NO. 6), at different concentrations (1.5 mg/ml, 1 mg/ml and 0.5 mg/ml) and the isoform of the peptide of the present disclosure, Peptide 6, ITRI-2, at different concentrations (1.5 mg/ml, 1 mg/ml and 0.5 mg/ml), IL-6 at different concentrations (1000 ng/ml and 100 ng/ml) and TGF-α (10 ng/ml), and MMP9 secretion of HaCaT cells was detected (HaCaT cells not stimulated were used as a blank group).

The experimental method is the same as above-mentioned item “5. Detection of MMP9 secretion of human keratinocyte cell line HaCaT stimulated with peptides” of “B. Method” paragraph.

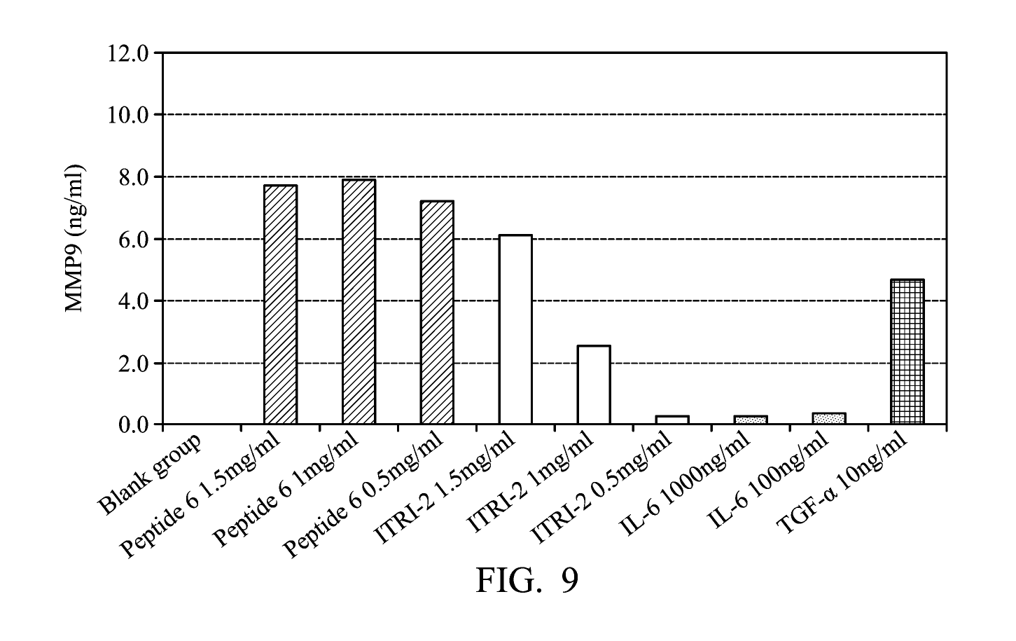

The results of stimulating human keratinocyte cell line HaCaT and determining MMP9 secretion thereof are shown in FIG. 9.

As illustrated in FIG. 9, the peptide of the present disclosure, Peptide 6, and an isoform thereof ITRI-2 are capable of inducing a higher level of MMP9 secretion than IL-6.

### Example 3

Analysis of the Effects of Short Form Peptides of a Peptide of the Present Disclosure

Within the range of the sequence of the peptide of the present disclosure, Peptide 6 (the sequence thereof was SEQ ID NO. 6), 3 short form peptides of the peptide of the present disclosure, Peptide 6, peptides 6M14, 6R21 and 6L21, were further designed.

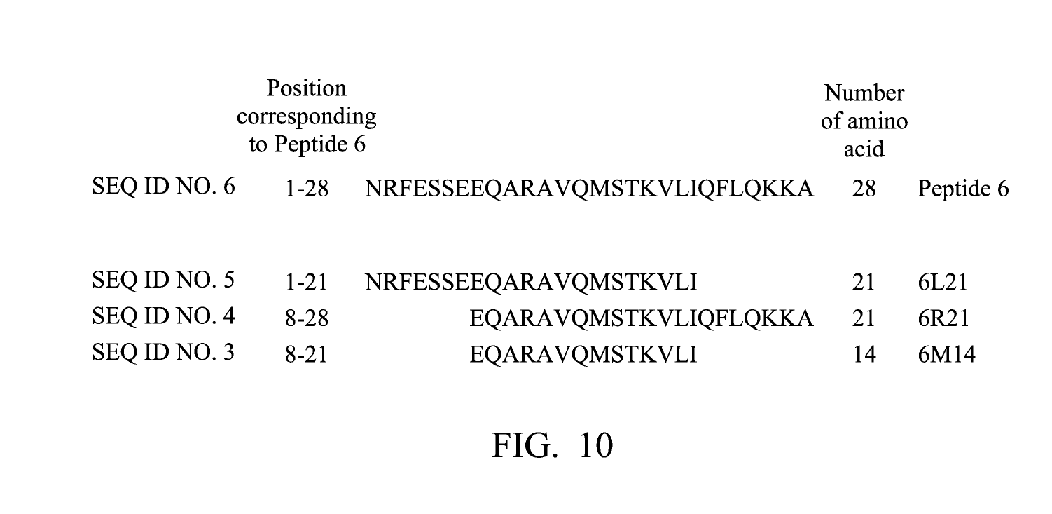

Sequence alignment for the sequence of each short form peptide and the sequence of the peptide of the present disclosure, Peptide 6 can refer to FIG. 10, and the sequence of each short form peptide is described as follows:

Sequence of 6M14: SEQ ID NO. 3;

Sequence of 6R21: SEQ ID NO. 4;

Sequence of 6L21: SEQ ID NO. 5.

1. Effects of Short Form Peptides of the Peptide of the Present Disclosure on Cell Migration of Human Keratinocyte Cell

In this experiment, the effects of short form peptides of the peptide of the present disclosure on cell migration of human keratinocyte cells were determined.

Human keratinocyte cell line HaCaT cells were stimulated with the peptide of the present disclosure, Peptide 6 (the sequence thereof was SEQ ID NO. 6) (1000 μg/ml) and different short form peptides of the peptide of the present disclosure, Peptide 6: peptide 6M14 (1000 μg/ml) and peptide 6L21 (1400 μg/ml) and TGF-α (10 ng/ml), and cell migration levels of the HaCaT cells were determined (HaCaT cells not stimulated with a peptide or growth factor were used as a control group).

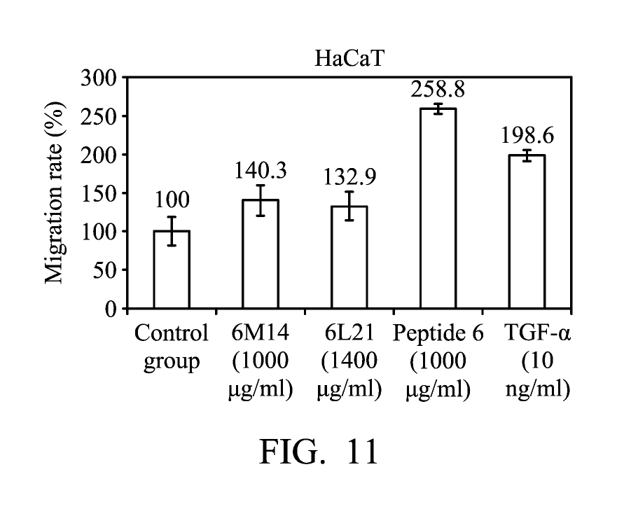

The experimental method is the same as above-mentioned item “4. Cell migration analysis of human keratinocyte cell line HaCaT stimulated with peptides” of “B. Method” paragraph. The results are shown in FIG. 11.

As illustrated in FIG. 11, the above-mentioned short form peptides of the peptide of the present disclosure, Peptide 6, are all capable of promoting cell migration, similar to Peptide 6.

2. Effects of Short Form Peptides of the Peptide of the Present Disclosure on TNF-α Secretion of Human Monocyte-Derived Macrophages

Macrophages obtained from the blood of Donor 5 were stimulated with the peptide of the present disclosure, Peptide 6 (the sequence thereof was SEQ ID NO. 6) (50 μg/ml), a short form peptides of the peptide of the present disclosure, Peptide 6, 6M14 (50 μg/ml), and IL-6 (200 ng/ml), and TNF-α secretion of macrophages was detected (macrophages not stimulated were used as a blank group).

The experimental method is the same as above-mentioned item “2. Detection of TNF-α secretion of monocyte-derived macrophages stimulated with peptides” of “B. Method” paragraph.

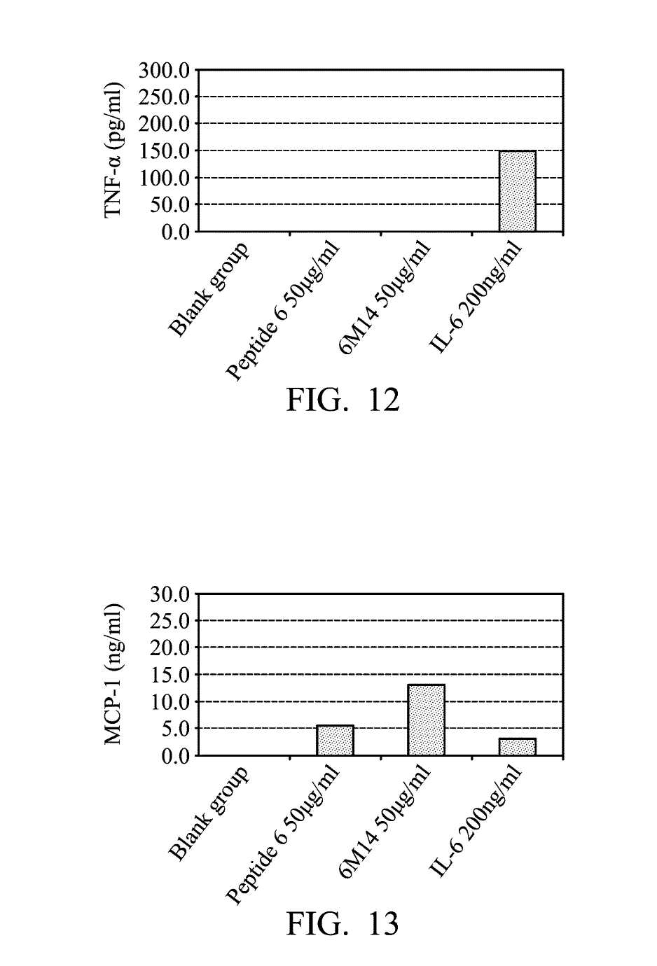

The results of stimulating macrophages of Donor 5 and determining TNF-α secretion thereof are shown in FIG. 12.

As illustrated in FIG. 12, the peptide of the present disclosure, Peptide 6, and a short form peptide thereof 6M14 almost do not induce TNF-α secretion, even at a concentration of 50 μg/ml, whereas even at a low concentration, IL-6 can still induce macrophages to secrete a great quantity of TNF-α.

3. Effects of Short Form Peptides of the Peptide of the Present Disclosure on MCP-1 Secretion of Human Monocyte-Derived Macrophages

Macrophages obtained from the blood of Donor 5 were stimulated with the peptide of the present disclosure, Peptide 6 (the sequence thereof was SEQ ID NO. 6) (50 μg/ml), short form peptides of the peptide of the present disclosure, Peptide 6, 6M14 (50 μg/ml), and IL-6 (200 ng/ml), and MCP-1 secretion of macrophages was detected (macrophages not stimulated were used as a blank group).

The experimental method is the same as above-mentioned item “3. Detections of MCP-1 secretion of human umbilical vein endothelial cells (HUVEC) and human monocyte-derived macrophages stimulated with peptides” of “B. Method” paragraph.

The results of stimulating macrophages of Donor 5 and determining MCP-1 secretion thereof are shown in FIG. 13.

As illustrated in FIG. 13, the peptide of the present disclosure, Peptide 6, and short form peptides thereof 6M14 are capable of inducing a higher level of MCP-1 secretion than IL-6. Moreover, the MCP-1 secretion level induced by the short form peptides of the peptide of the present disclosure, Peptide 6, is higher than that of the peptide of the present disclosure, Peptide 6.

It will be apparent to those skilled in the art that various modifications and variations can be made to the disclosed embodiments. It is intended that the specification and examples be considered as exemplary only, with the true scope of the disclosure being indicated by the following claims and their equivalents.

What is claimed is:

1. A peptide for promoting cell migration and/or skin wound healing, comprising:
an IL-6-derived peptide, which is designed within the region of the sequence of SEQ ID NO. 1 and of which the sequence comprises the sequence of SEQ ID NO. 2, wherein the IL-6-derived peptide has about 6-50 amino acids.

2. The peptide for promoting cell migration and/or skin wound healing as claimed in claim 1, wherein the sequence of the IL-6-derived peptide is the sequence of SEQ ID NO. 2.

3. The peptide for promoting cell migration and/or skin wound healing as claimed in claim 1, wherein the sequence of the IL-6-derived peptide comprises the sequence of SEQ ID NO. 3, the sequence of SEQ ID NO. 4, the sequence of SEQ ID NO. 5 or the sequence of SEQ ID NO. 6.

4. The peptide for promoting cell migration and/or skin wound healing as claimed in claim 1, wherein the IL-6-derived peptide has about 14-28 amino acids.

5. The peptide for promoting cell migration and/or skin wound healing as claimed in claim 4, wherein the sequence of the IL-6-derived peptide is the sequence of SEQ ID NO. 3, the sequence of SEQ ID NO. 4, the sequence of SEQ ID NO. 5 or the sequence of SEQ ID NO. 6.

6. The peptide for promoting cell migration and/or skin wound healing as claimed in claim 1, wherein the IL-6-derived peptide has at least one D-form amino acid.

7. The peptide for promoting cell migration and/or skin wound healing as claimed in claim 6, wherein the position of the at least one D-form amino acid is one or more selected from the group consisting of the following:
(a) the first amino acid in front of the at least one metalloproteinase cleavage site of the IL-6-derived peptide;
(b) the first amino acid in front of the at least one serine proteases cleavage site of the IL-6-derived peptide; and
(c) the first amino acid in front of the at least one cleavage site for another enzyme existing in a wound of the IL-6-derived peptide.

8. The peptide for promoting cell migration and/or skin wound healing as claimed in claim 6, wherein the sequence of the IL-6-derived peptide comprises the sequence of SEQ ID NO. 6.

9. The peptide for promoting cell migration and/or skin wound healing as claimed in claim 6, wherein the at least one D-form amino acid is one or more of the following:
(a) alanine at position 12 of the sequence of SEQ ID NO. 6;
(b) valine at position 13 of the sequence of SEQ ID NO. 6;
(c) glutamine at position 14 of the sequence of SEQ ID NO. 6;
(d) methionine at position 15 of the sequence of SEQ ID NO. 6;
(e) valine at position 19 of the sequence of SEQ ID NO. 6;
(f) isoleucine at position 21 of the sequence of SEQ ID NO. 6; and
(g) phenylalanine at position 23 of the sequence of SEQ ID NO. 6.

10. The peptide for promoting cell migration and/or skin wound healing as claimed in claim 8, wherein the sequence of the IL-6-derived peptide is the sequence of SEQ ID NO. 6, and the at least one D-form amino acid is alanine at position 12 of the sequence of SEQ ID NO. 6, valine at position 13 of the sequence of SEQ ID NO. 6, glutamine at position 14 of the sequence of SEQ ID NO. 6, methionine at position 15 of the sequence of SEQ ID NO. 6, valine at position 19 of the sequence of SEQ ID NO. 6, isoleucine at position 21 of the sequence of SEQ ID NO. 6 or phenylalanine at position 23 of the sequence of SEQ ID NO.

11. A pharmaceutical composition for promoting cell migration and/or skin wound healing, comprising:
an IL-6-derived peptide, which is designed within the region of the sequence of SEQ ID NO. 1 and of which the sequence comprises the sequence of SEQ ID NO. 2, wherein the IL-6-derived peptide has about 6-50 amino acids; and
a pharmaceutically acceptable carrier or salt.

12. The pharmaceutical composition for promoting cell migration and/or skin wound healing as claimed in claim 11, wherein the IL-6-derived peptide has about 14-28 amino acids.

13. The pharmaceutical composition for promoting cell migration and/or skin wound healing as claimed in claim 12, wherein the sequence of the IL-6-derived peptide is the sequence of SEQ ID NO. 3, the sequence of SEQ ID NO. 4, the sequence of SEQ ID NO. 5 or the sequence of SEQ ID NO. 6.

14. The pharmaceutical composition for promoting cell migration and/or skin wound healing as claimed in claim 11, wherein the IL-6-derived peptide has at least one D-form amino acid.

15. The pharmaceutical composition for promoting cell migration and/or skin wound healing as claimed in claim 14, wherein the sequence of the IL-6-derived peptide is the sequence of SEQ ID NO. 6, and the at least one D-form amino acid is alanine at position 12 of the sequence of SEQ ID NO. 6, valine at position 13 of the sequence of SEQ ID NO. 6, glutamine at position 14 of the sequence of SEQ ID NO. 6, methionine at position 15 of the sequence of SEQ ID NO. 6, valine at position 19 of the sequence of SEQ ID NO. 6, isoleucine at position 21 of the sequence of SEQ ID NO. 6 or phenylalanine at position 23 of the sequence of SEQ ID NO. 6.

16. A method for promoting cell migration and/or skin wound healing, comprising:
administering an effective amount of the pharmaceutical composition for promoting cell migration and/or skin wound healing as claimed in claim 11 to a subject in need thereof to promote cell migration and/or skin wound healing.


SEQUENCE LISTING (6 sequences)

SEQ ID NO: 1 - protein, 183 aa, Homo sapiens
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 6 aa, Artificial Sequence
  Artificially Synthesized Peptide
AVQMST

SEQ ID NO: 3 - protein, 14 aa, Artificial Sequence
  Artificially Synthesized Peptide
EQARAVQMSTKVLI

SEQ ID NO: 4 - protein, 21 aa, Artificial Sequence
  Artificially Synthesized Peptide
EQARAVQMSTKVLIQFLQKKA

SEQ ID NO: 5 - protein, 21 aa, Artificial Sequence
  Artificially Synthesized Peptide
NRFESSEEQARAVQMSTKVLI

SEQ ID NO: 6 - protein, 28 aa, Artificial Sequence
  Artificially Synthesized Peptide
NRFESSEEQARAVQMSTKVLIQFLQKKA